(19) United States
(12) Patent Application Publication
(10) Pub. No.: US 2026/0076851 A1
(43) Pub. Date: Mar. 19, 2026

(54) CONTROL SYSTEM FOR A PATIENT SUPPORT APPARATUS

(71) Applicant: Hill-Rom Services, Inc., Batesville, IN (US)

(72) Inventors: Scott M. Corbin, Sunman, IN (US); Edward J. Koors, Indianapolis, IN (US); Jonathan K. Moenter, Batesville, IN (US); Gavin M. Monson, Oxford, OH (US); Unnati Ojha, Apex, NC (US); Chelsea Olson Riehle, Batesville, IN (US); Jonathan D. Turner, Dillsboro, IN (US); Bryan Matthew Weidman, Columbus, IN (US); David C. Newkirk, Lawrenceburg, IN (US); Brian Guthrie, Greensburg, IN (US); Jeffrey S. Jelinek, Oldenburg, IN (US); David P. Laloge, Batesville, IN (US); Frank E. Sauser, Cincinnati, OH (US); Douglas E. Borgman, Brookville, IN (US); Chris L. Hildenbrand, Osgood, IN (US); Brandon P. Fisk, Brookville, IN (US); Douglas A. Seim, Okeana, OH (US)

(73) Assignee: Hill-Rom Services, Inc., Batesville, IN (US)

(21) Appl. No.: 19/399,745

(22) Filed: Nov. 25, 2025

Related U.S. Application Data
(63) Continuation-in-part of application No. 18/680,889, filed on May 31, 2024.
(63) Continuation-in-part of application No. 19/008,273, filed on Jan. 2, 2025.
(63) Continuation-in-part of application No. 29/992,474, filed on Mar. 8, 2025.
(63) Continuation-in-part of application No. 19/271,229, filed on Jul. 16, 2025.
(63) Continuation-in-part of application No. PCT/IB2025/053201, filed on Mar. 26, 2025.
(63) Continuation-in-part of application No. 30/018,914, filed on Aug. 19, 2025.
(63) Continuation-in-part of application No. 30/018,913, filed on Aug. 19, 2025.
(63) Continuation-in-part of application No. 30/018,911, filed on Aug. 19, 2025.
(60) Provisional application No. 63/505,196, filed on May 31, 2023.
(60) Provisional application No. 63/621,448, filed on Jan. 16, 2024.
(60) Provisional application No. 63/571,191, filed on Mar. 28, 2024.
(60) Provisional application No. 63/643,397, filed on May 6, 2024.
(60) Provisional application No. 63/758,551, filed on Feb. 14, 2025.
(60) Provisional application No. 63/769,453, filed on Mar. 10, 2025.
(60) Provisional application No. 63/830,570, filed on Jun. 26, 2025.
(60) Provisional application No. 63/845,071, filed on Jul. 16, 2025.
(60) Provisional application No. 63/908,241, filed on Oct. 30, 2025.

(51) Int. Cl. A61G 7/018; A61G 7/015; A61G 12/00; G16H 40/63

(52) U.S. Cl. CPC A61G 7/018; A61G 7/015; A61G 12/008; G16H 40/63; A61G 2203/10; A61G 2203/16; A61G 2203/20

(57) ABSTRACT

A patient support apparatus includes a control system that simplifies operation of the patient support apparatus for a caregiver by simplifying the information available to the caregiver during operation of the patient support apparatus. The patient support apparatus provides indicators at the site of care for the caregiver, as well as indicators that are discernable from a distance, indicators providing concise information to the caregiver regarding the status of functions of the patient support apparatus and an alarm function of the patient support apparatus.

### CROSS-REFERENCE TO RELATED APPLICATIONS

This application claims the benefit of:

U.S. patent application Ser. No. 18/680,889, filed May 31, 2024, which claims priority under 35 U.S. C. § 119(e) to U.S. Provisional Application Ser. No. 63/505,196, filed May 31, 2023, and, to U.S. Provisional Application Ser. No. 63/643,397, filed May 6, 2024;

this application also claims the benefit of U.S. patent application Ser. No. 19/008,273, filed Jan. 1, 2025, which claims priority under 35 U.S. C. § 119(e) to U.S. Provisional Application Ser. No. 63/621,448, filed Jan. 16, 2024;

this application also claims priority under 35 U.S.C. § 119(e) to U.S. Provisional Application Ser. No. 63/758,551, filed Feb. 14, 2025;

this application also claims the benefit of U.S. Design Patent Application Ser. No. 29/992,474, filed Mar. 8, 2025;

this application also claims priority to U.S. patent application Ser. No. 19/271,229, which claims the benefit under 35 U.S. C. § 119(e) of U.S. Provisional Application Ser. No. 63/673,386, filed Jul. 19, 2024 and U.S. Provisional Application Ser. No. 63/768,944, filed Mar. 8, 2025;

this application also priority under 35 U.S.C. § 119(e) to U.S. Provisional Application Ser. No. 63/769,453, filed Mar. 10, 2025;

this application also claims priority to PCT International Application No. PCT/IB2025/053201, filed Mar. 26, 2025, which claims the benefit U.S. Provisional Application Ser. No. 63/571,191, filed Mar. 28, 2024;

this application also claims priority under 35 U.S.C. § 119(e) to U.S. Provisional Application Ser. No. 63/830,570, filed Jun. 26, 2025;

this application also claims priority under 35 U.S.C. § 119(e) to U.S. Provisional Application Ser. No. 63/845,071, filed Jul. 16, 2025;

this application also claims priority to U.S. Design Patent Application Ser. No. 30/018,914, filed Aug. 19, 2025;

this application also claims priority to U.S. Design Patent Application Ser. No. 30/018,913, filed Aug. 19, 2025;

this application also claims priority to U.S. Design Patent Application Ser. No. 30/018,911, filed Aug. 19, 2025; and

this application also claims priority under 35 U.S. C. § 119(e) to U.S. Provisional Application Ser. No. 63/908,241, filed Oct. 30, 2025;

each of which all of which are expressly incorporated by reference herein.

### BACKGROUND

The present disclosure is directed to a control system for a patient support apparatus. More specifically, the present disclosure is related to the user interface of a patient support apparatus that improves the user experience for a caregiver.

In the modern healthcare facility, patients are often kept for extended periods in the emergency unit or an observation ward on patient support apparatus while tests are run and the patient is under observation. Patient support apparatuses (i.e., beds and stretchers) typically have manual articulation controls that require pulling one of two or more handles to release a locked component so that a portion of the patient support apparatus's frame can be articulated. For example, pulling a handle on the top corner of the stretcher's head section may release the head section's locking gas spring so that the head section could be raised or lowered. The challenge with these articulation control systems is designing them so that the patient support apparatus can be articulated by using either one handle at a time or both handles at the same time. The control systems get complex, are costly, and have multiple adjustment points for tuning the actuation stroke of the total system.

In some instances, the patient may be in significant discomfort, lack mobility, or be otherwise incapacitated. As such, there may be a need to articulate the patient support apparatus based on the patient's acuity level. Thus, there is a need for a patient support apparatus structure that has common elements for basic operation, but that can be articulated easily.

Additionally, as the patients are often kept for extended periods in the emergency unit or an observation ward, they may need to be transported to have tests or procedures done in other parts of a facility. It is known to have battery powered components on the patient support apparatuses used for transporting to assist with positioning the patient and to power drive the patient support apparatus to reduce the load on the caregiver staff. While the caregiver staff may wish to rely on the powered components, failure to plug a patient support apparatus to mains power once it has been moved results in batteries being dissipated. This drives a need for a caregiver to be able to readily ascertain the charges status of a patient support apparatus. It is especially important for the caregiver to understand when the battery level prevents the patient support apparatus from operating properly, such as when alarm function is inoperable due to a battery charge level.

Additionally, the transport process is cumbersome when cords and lines are draped over the patient support apparatus. This drives the need for line and cord management structures that may be used when the patient support apparatus is in transport.

### SUMMARY

The present disclosure includes one or more of the features recited in the appended claims and/or the following features which, alone or in any combination, may comprise patentable subject matter.

According to a first aspect of the present disclosure, a patient support apparatus comprises a control system comprising a power supply connectable to mains power, a battery, a battery charger, and an indication system, the indication system comprising an indicator providing the status of the battery charger and an indication of the level of charge of the battery.

In some embodiments of the first aspect, the indication system includes a battery charge indicator positioned at a foot end of the patient support apparatus, the battery charge indicator being illuminated in multiple states to provide a status of the battery charger.

In some embodiments of the first aspect, the battery charge indicator provides an indication of the level of the charge of the battery and the battery charger status simultaneously.

In some embodiments of the first aspect, the control system includes a system on a module that has a power reduction mode that is automatically engaged through an operating system when the patient support apparatus is operated on battery power without any activity for a predetermined period of time.

In some embodiments of the first aspect, the indication system includes a battery charge indicator positioned at a foot end of the patient support apparatus, the battery charge indicator being illuminated in multiple states to provide an indication of the level of the charge of the battery.

In some embodiments of the first aspect, the indication system includes a battery charge indicator positioned at a foot end of the patient support apparatus, the battery charge indicator being illuminated in multiple states to provide an indication of whether the bed is connected to mains power.

In some embodiments of the first aspect, the control system further comprises a graphical user interface, the graphical user interface configured to provide a battery status indicator, the indicator having multiple display states that provide an indication of the level of charge of the battery.

In some embodiments of the first aspect, the battery status indicator indicates whether the battery is being charged.

In some embodiments of the first aspect, the multiple display states include incremental charge levels represented.

In some embodiments of the first aspect, the graphical user interface is operable to generate an alert to indicate that the battery charge level is insufficient to maintain operation of a patient alarm function.

In some embodiments of the first aspect, the indicator is illuminated on a component of the bed and is simultaneously projected on a floor adjacent the patient support apparatus.

According to a second aspect of the present disclosure, a control system for a patient support apparatus is operable to provide an indication of the status of a battery of the patient support apparatus, the status of the battery including whether the battery is being charged, whether the battery is low in charge, and whether the battery is fully charged.

In some embodiments of the second aspect, the indication is provided on a graphical user interface.

In some embodiments of the second aspect, the control system further provides an indication that the operation of a critical function of the patient support apparatus cannot be supported by the level of charge of the battery. In some embodiments of the second aspect, the indication that the operation of the critical function cannot be supported by the level of charge of the battery is provided on a graphical user interface.

In some embodiments of the second aspect, a user is prompted to modify the operation of the patient support apparatus to mitigate the support of the critical function.

According to a third aspect of the present disclosure, a patient support apparatus comprises a base assembly, a lift arm assembly, a deck assembly, and a control system. The lift arm assembly is coupled to the base assembly and pivotable relative to the base assembly at a head end of the base assembly. The deck assembly is supported on the lift arm assembly, the deck assembly including, a head deck a movable relative to the lift arm assembly, thigh deck section movable relative to the lift arm assembly, and a foot deck section pivotably coupled to the thigh deck section, the foot deck section having a distal end spaced apart from the thigh deck section. The control system includes a first user input operable, when activated by a user, to cause the head deck section to be powered to be moved to a lowered position, the thigh deck section to be powered to be moved to a raised position, and the distal end of the foot deck section to be lowered relative to the thigh deck section.

In some embodiments of the third aspect, the operation of the first user input may be locked out.

In some embodiments of the third aspect, the patient support apparatus further comprises a graphical user interface, the first user input being displayed on a touchscreen of the graphical user interface.

In some embodiments of the third aspect, the control system includes a second user input operable, when activated by a user, to cause the thigh deck section to be powered to a lowered position and the distal end of the foot deck section to be raised to thereby place the head deck section, thigh deck section, and foot deck section in a flat position.

In some embodiments of the third aspect, the control system includes a third user input operable, when activated by a user when activated by a user, to cause the head deck section to be powered to be moved to a raised position, the thigh deck section to be powered to be moved to a raised position, and the distal end of the foot deck section to be lowered relative to the thigh deck section.

In some embodiments of the third aspect, the control system is operable to provide an indication of the status of a battery of the patient support apparatus, the status of the battery including whether the battery is being charged, whether the battery is low in charge, and whether the battery is fully charged.

In some embodiments of the third aspect, the control system further provides an indication that the operation of a critical function of the patient support apparatus cannot be supported by the level of charge of the battery.

In some embodiments of the third aspect, the indication is provided on a graphical user interface.

In some embodiments of the third aspect, a user is prompted to modify the operation of the patient support apparatus to mitigate the support of the critical function.

In some embodiments of the third aspect, the patient support apparatus further comprises a mattress having a weight positioned to maintain the mattress in contact with the foot deck section when the thigh deck section is raised and the distal end of the foot deck section is lowered.

In some embodiments of the third aspect, the lift arm assembly is driven by an electrically powered hydraulic motor, the motor being engaged when a user pushes a foot pedal down to engage an electric switch.

In some embodiments of the third aspect, the foot pedal includes an indicator that is illuminated to indicate that the electrically powered hydraulic motor is present.

In some embodiments of the third aspect, the patient support apparatus has a central wheel for steering the patient support apparatus over the floor.

In some embodiments of the third aspect, the patient support apparatus further comprises a push handle that includes a grip that rotates relative to a shaft of the push handle.

In some embodiments of the third aspect, the central wheel includes a motor for deploying the wheel into engagement with a floor and a drive motor for causing the wheel to be rotated to drive the patient support apparatus over the floor.

In some embodiments of the third aspect, the patient support apparatus further comprises an oxygen tank holder positioned at a head end of the patient support apparatus.

In some embodiments of the third aspect, the patient support apparatus further comprises comprising a power cord having a plug and prongs extending from the plug and a circular receptacle formed in the patient support apparatus and sized to receive the prongs in the circular receptacle with an interference fit to secure the plug to the circular receptacle.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of a patient support apparatus according to the present disclosure, the patient support apparatus viewed from the patient's left foot end and positioned in a chair configuration with a head deck section and thigh deck section raised and a distal end of a foot deck section being lowered relative to the thigh deck section;

FIG. 2 is a side view of the patient support apparatus of FIG. 1 looking from the patient's left side of the patient support apparatus, the patient support apparatus being positioned in a pelvic position;

FIG. 3 is a view similar to FIG. 2, with portions omitted, the patient support apparatus moved to a tilted position with the foot end of an upper frame being lowered relative to the head end of the upper frame to achieve a deeper chair position;

FIG. 4 is an enlarged view of a portion of the outboard side of a patient's left siderail of the patient support apparatus of FIGS. 1-3, FIG. 4 showing the relationship of a key panel and a graphical user interface supported on the siderail;

FIG. 5 is an enlarged view of the key panel of FIG. 4;

FIG. 6 is an enlarged view of the graphical user interface of FIG. 4;

FIG. 7 is a menu tree depicting the various functions available on the graphical user interface of FIG. 6;

FIG. 8 is a view of a typical home screen shown on the graphical user interface of FIG. 6;

FIG. 9 is a view of a screen that is displayed on the graphical user interface when a pause alarm function is selected on the home screen of FIG. 8;

FIG. 10 is a view of a typical home screen, similar to the home screen shown in FIG. 8, the home screen of FIG. 10 showing different information;

FIG. 11 is a graphical representation of a battery icon displayed on the home screens of FIGS. 8 and 10, the battery icon indicating a full charge;

FIG. 12 is a graphical representation of a battery icon displayed on the home screens of FIGS. 8 and 10, similar to FIG. 11, the battery icon indicating a full charge and the battery being charged as indicated by a charging indicator overlying the battery;

FIG. 13 is a graphical representation of a battery icon displayed on the home screens of FIGS. 8 and 10, similar to FIGS. 11 and 12, the battery icon indicating a critically low charge as indicated by single bar and the exclamation mark overlying the battery;

FIG. 14 is a view of a screen that is displayed on the graphical user interface when the battery is critically low and the patient support apparatus has a alarm function active;

FIG. 15 is a graphical representation of a patient facing control positioned on the inboard side of the patient's right siderail in FIG. 1;

FIG. 16 is a view of a screen that is displayed on the graphical user interface when the stretcher is not in a level condition and unable to take a scale reading;

FIG. 17 is a view of a screen that is displayed on the graphical user interface when the stretcher is not in a level condition and not in an optimum position to take a scale reading;

FIG. 18 is a view of a screen that is displayed on the graphical user interface when a patient has been identified to be associated with the patient support apparatus, the screen providing inputs for a caregiver/user to confirm the patient identity or take alternative action;

FIG. 19 is a view of a screen that is displayed on the graphical user interface when a patient has been identified to be associated with the patient support apparatus, the patient's weight has been taken, and the weight has been transferred to an electronic medical record;

FIG. 20 is a view of a screen that is displayed on the graphical user interface when new software has been identified as being available to update the control system of the patient support apparatus;

FIG. 21 is a view of a screen that is displayed on the graphical user interface when the software is updating;

FIG. 22 is a view of a screen that is displayed on the graphical user interface when the software update is complete;

FIG. 23 is a perspective view of the foot end of the patient support apparatus of FIG. 1 showing the operation of indicators at the foot end of the patient support apparatus;

FIG. 24 is a head end view of an embodiment of the patient support apparatus with a power cord stored on a power cord holder;

FIG. 25 is a view of a plug power cord positioned in a multi-purpose receiver at the head end of the patient support apparatus, the receiver having a power cord engaged with the receiver to stow the power cord during transport of the patient support apparatus;

FIG. 26 is a perspective exploded assembly drawing showing the connection of a weigh frame of the patient support apparatus to an upper frame of the patient support apparatus; and

FIG. 27 is a block diagram of the control system of the patient support apparatus of FIGS. 1-3.

### DETAILED DESCRIPTION

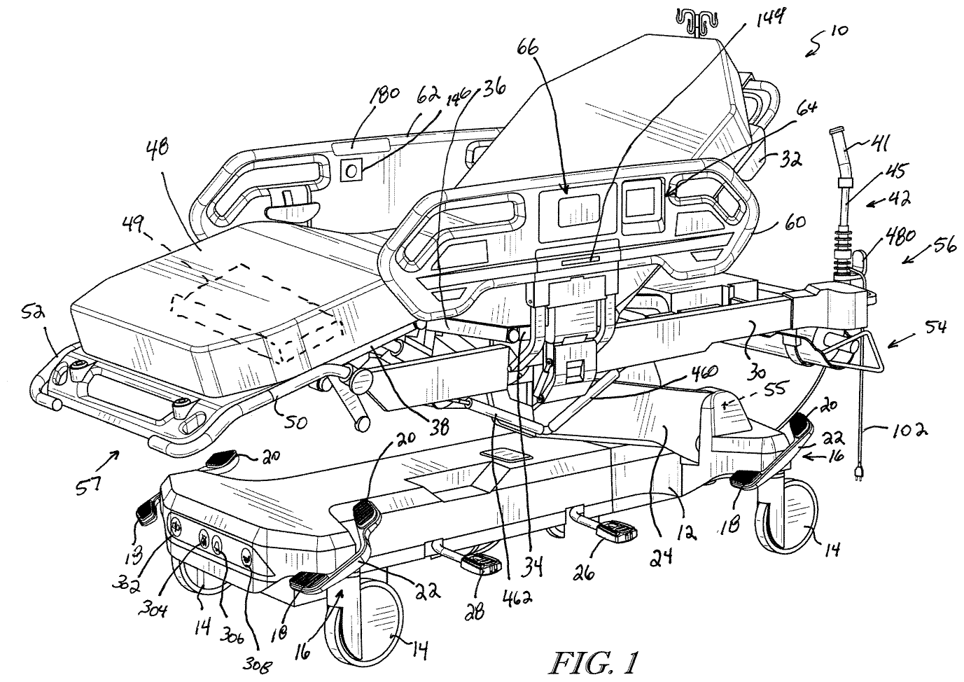

The present disclosure is directed to a patient support apparatus 10, illustratively embodied as a procedural stretcher 10, as shown in FIG. 1. The patient support apparatus 10 includes a base assembly 12 supported by four casters 14, the casters 14 operable to rotate and swivel to allow the patient support apparatus 10 to move over a floor. As shown in FIG. 1, the casters 14 are secured in a locked position with the pedal assembly 16 positioned at each of the casters 14. Each pedal assembly 16 has two foot pads 18, 20, the foot pads being actuable by a caregiver to move the pedal assembly 16 between three positions. In a first position shown in FIG. 1, a foot pad 18 has been depressed to place the casters 14 in a locked position whereby all four of the casters 14 are secured to prevent movement and thereby place the patient support apparatus 10 in a locked position. Actuation of any one of the foot pedal assemblies 16 controls an actuation mechanism for all four of the casters 14 and each pedal assembly 16 moves with the actuation mechanism when any one of the pedal assemblies 16 is actuated. When the foot pad 20 is depressed, the foot pedal assembly 16 will rotate to place the foot pad 20 in a lowered position engaging a steer mode of the casters 14 such that the patient support apparatus 10 is movable with a single caster 14 locked to prevent swiveling about the stem, as is known in the art. When a foot pedal assembly 16 is placed in a neutral position where neither foot pad 18 or 20 is lowered and a crossbar 22 is level, the casters 14 are in a neutral position where all of the casters 14 are free to swivel and rotate. In some embodiments, the steer mode will deploy a central wheel from the base assembly 12 as will be described below.

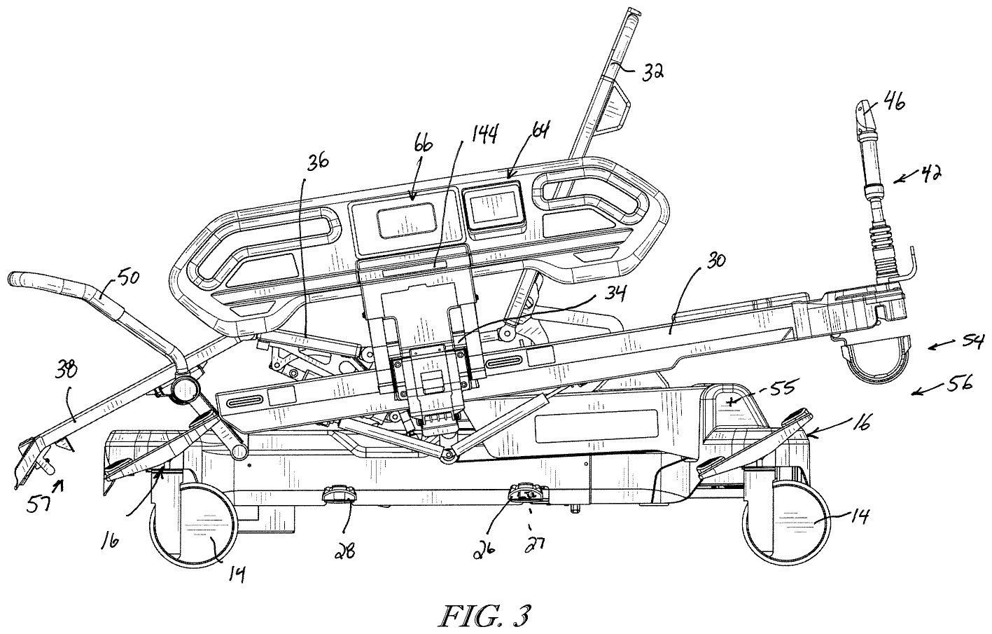

The base assembly 12 supports a lift arm assembly 24 which may be actuated to move between a lowered position such as that shown in FIG. 3 and the raised position shown in FIG. 1. The lift arm assembly 24 is moved from the lowered position to the raised position by activation of a foot pedal 26 which operates a hydraulic cylinder (not shown). The lift arm assembly 24 is pivotable relative to the base assembly 12 about an axis 55 at a head end 56 of the patient support apparatus 10. The foot pedal 26 may be used to move hydraulic fluid into the hydraulic cylinder manually and/or may be used activate a hydraulic pump to move hydraulic fluid into the hydraulic cylinder. The operation of the mechanisms used to cause the lift arm assembly 24 to rise are described in detail in U.S. provisional patent application No. 63/908,241 titled “PATIENT SUPPORT APPARATUS HAVING HYDRAULIC LIFT PEDAL SYSTEM,” which is incorporated by refence in its entirety for the description of the related structures. The patient support apparatus 10 also includes a foot pedal 28 that is depressed to cause the hydraulic fluid in the hydraulic cylinder to be released so that the lift arm assembly 24 is lowered from a raised position.

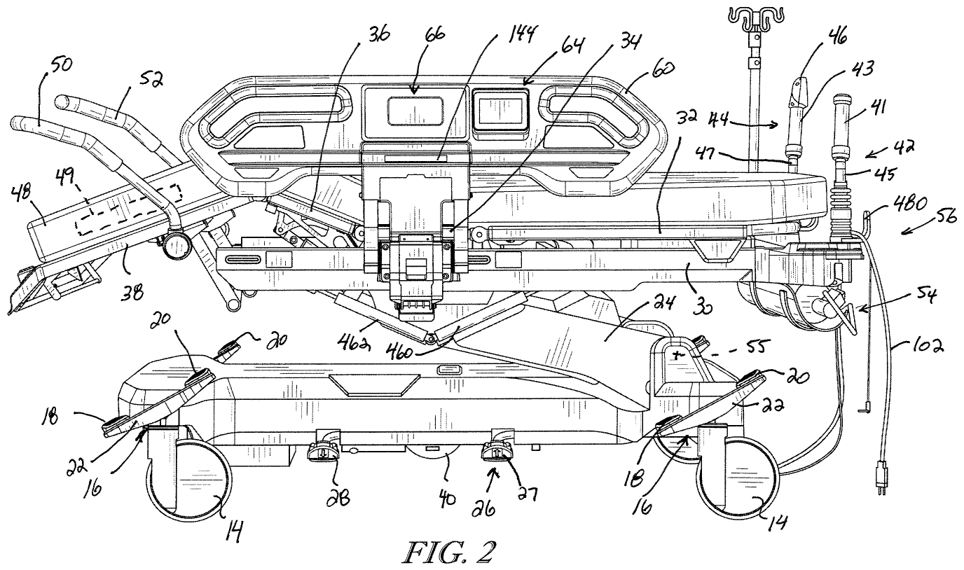

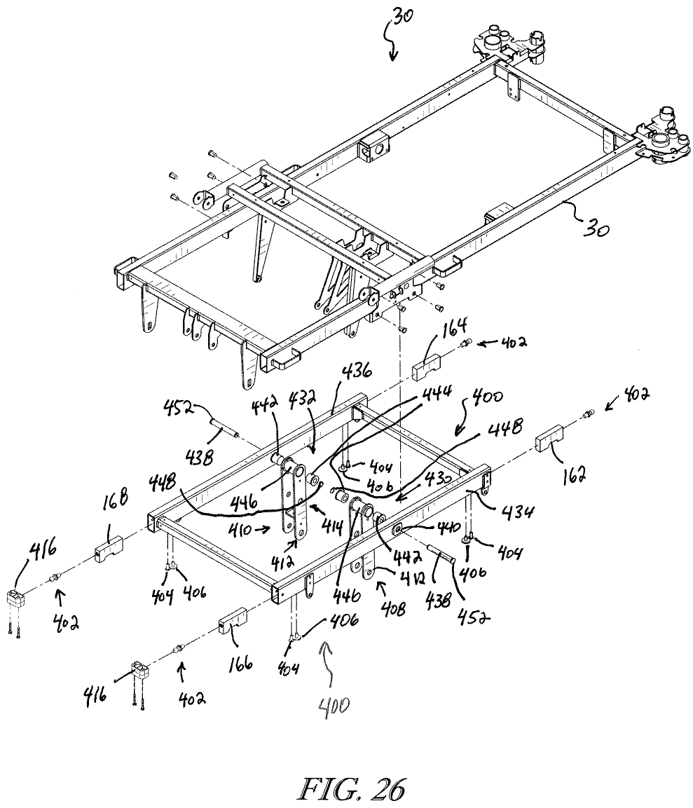

The lift arm assembly 24 supports a weigh frame 400 (best seen in FIG. 26) that supports an upper frame 30. The upper frame 30 supports a head deck section 32, a seat deck section 34, a thigh deck section 36 and a foot deck section 38, the head deck section 32, thigh deck section 36, and foot deck section 38 articulated and movable relative to the upper frame 30. Because the lift arm assembly 24 pivots relative to the base assembly 12 at the head end 56, a foot end 57 of the upper frame 30 and foot deck section 38 are cantilevered relative to the base assembly 12 to provide clearance under thigh deck section 36 and the foot deck section 38. In addition, the upper frame 30 is pivotable relative to the lift arm assembly 24 as suggested in FIG. 3. The arrangement of the lift arm assembly 24 to the base assembly 12 allows the patient support apparatus 10 to achieve the unique pelvic examination position of FIG. 2. The movement of the upper frame relative to the lift arm assembly 24 and the head deck section 32, thigh deck section 36, and foot deck section 38 relative to the upper frame 30 is described in U.S. Patent Application Publication No. 2024/0398641A1, filed May 31, 2024 which is incorporated by refence in its entirety for the description of the related structures.

Referring now to FIG. 26, the weigh frame 400 engages four load beams 162, 164, 166, 168. The load beam 162 is positioned at the patient left head end of the weigh frame 400, the load beam 164 is positioned at the patient right head end of the of the weigh frame 400, the load beam 166 is positioned at the patient left foot end of the weigh frame 400, and the load beam 168 is positioned at the patient right foot end of the weigh frame 400. Each of the load beams 162, 164, 166, 168 are engaged by a respective ball end 402 that extends from each of the four load beams 162, 164, 166, 168 and supports the upper frame 30. The ball ends 402 are structured to support the upper frame 30 through a tilting of the upper frame 30 and transfer the load to the four load beams 162, 164, 166, 168 to be read and used as part of the scale function 206.

The load beams 162, 164, 166, 168 are each secured to the weigh frame 400 by a pair of the fasteners 404, 406. The weigh frame 400 is supported by a pair of link arms 408, 410 which engage with the lift arm assembly 24. The lift arm assembly 24 engages with each link arm 408, 410 at respective pivotable connections 412, 414 which correspond to the pivots 326 and 328 disclosed in US20230107717A1 which is titled “PATIENT SUPPORT APPARATUS WITH A HYDRAULIC LIFT HAVING POWERED AND MANUAL MODES,” which published Apr. 6, 2023 and which is incorporated herein for the discussion of the motion of the lift arm assembly 24.

A pair of foot end support blocks 416, 416 engage the ball ends 402, 402 of associated with load beam 166, 168 and are secured to the underside of a crossbar 420 of the upper frame 30. The ball ends 402, 402, of load beams 162, 164 engage support bearings (not shown) that are received in bearing housings 422, 424 of the upper frame 30. In this way, the weigh frame 400 is nested within the upper frame 30 and all of the weight supported by the upper frame 30 is transferred to the respective load beams 162, 164, 166, 168. The load beams 162, 164, 166, 168 support the support bearings and support blocks 416, 416 in a manner similar to that disclosed in U.S. Pat. No. 6,924,441, titled “LOAD CELL APPARATUS,” which incorporated by reference herein for the disclosure of the engagement of the ball ends 402 with the support bearings and support blocks 416, 416.

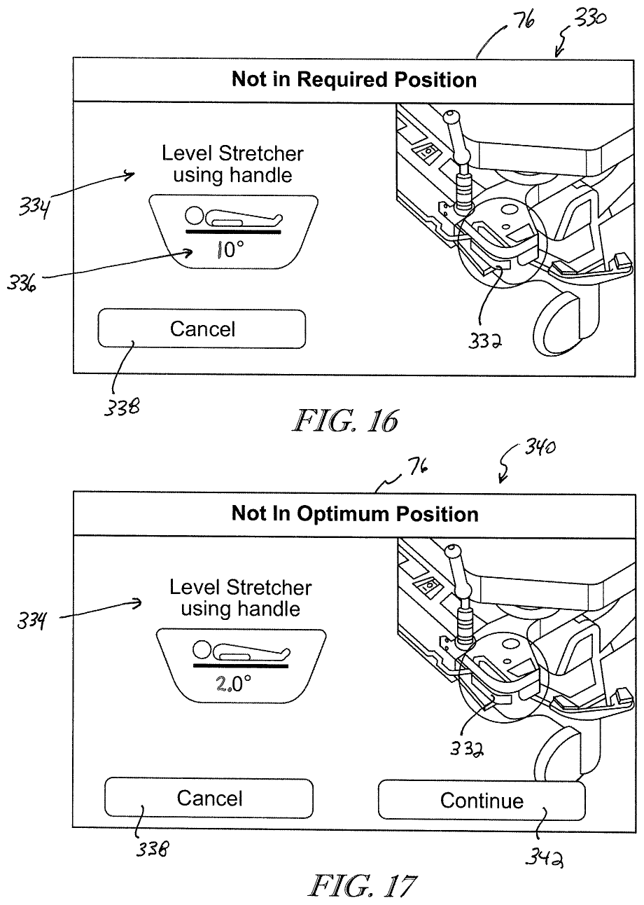

Tilting of the weigh frame 400 and, thereby, the upper frame 30 relative to the lift arm assembly 24 is accomplished through a pair of pivotable connection assemblies 430, 432 between the pair of link arms 408, 410 and respective side beams 434, 436 of the weigh frame 400. The pivotable connection assemblies 430, 432 each include a pivot pin 438 received through a fixed bearing 440 secured to the respective side beams 434, 436. The pivotable connection assemblies 430, 432 include an outer bearing 442 and an inner bearing 444 that are each received in cross-tube 446 of the respective link arms 408, 410 with the respective pivotable connection assembly 430 or 432 being secured by a respective snap-ring 448 secured to the inner end 450 of the respective pivot pin 438. The pivot pin 438 is formed with an enlarged head 452 that secures the pivot pin 438 to the exterior of the fixed bearing 440. In this way, the link arms 408, 410 are pivotable about an axis that is defined by the pivot pins 438. Pivoting of the weigh frame 400 and upper frame 30 is controlled by the release of the manual release 332 discussed with respect to FIGS. 16 and 17 below, which releases two pairs of releasable cylinders 460, 462 shown in FIGS. 2 and 3 which are connected to the pivots 326 of the link arms 408, 410 and the upper frame 30 to limit motion of the upper frame 30 and weigh frame 400 relative to the link arms 408, 410. When released, one of the cylinders 460, 462 extends while the other retracts, allowing for relative movement of the weigh frame 400 about the pivot pin 438.

In addition, the patient support apparatus 10 may further include a powered or unpowered wheel system that is deployed to cause a wheel 40 (see FIG. 3) to engage the floor to assist with steering the patient support apparatus 10 over the floor. A pair of push handles 42, 44, seen best in FIG. 2, are used to manually control the direction of the patient support apparatus 10 and a user interface 46 is positioned on the push handle 44 of the patient support apparatus 10. The user interface 46 provides inputs for a user to control the speed of the patient support apparatus 10 over the floor, when the powered version of the wheel system is used. The powered wheel system is described in detail in U.S. Provisional Application Ser. No. 63/845,071, filed Jul. 16, 2025 which is incorporated by refence in its entirety for the description of the related structures. The non-powered wheel system is described in U.S. Provisional Application Ser. No. 63/830,570, filed Jun. 26, 2025 which is incorporated by refence in its entirety for the description of the related structures. In some embodiments, when a non-powered wheel system is used, the respective grips 41, 43 of push handles 42, 44 rotate relative to the respective shafts 45, 47.

The patient support apparatus 10 further includes a mattress 48 as shown in FIG. 1. The mattress 48 includes a weight 49, shown in dotted lines in FIG. 1. One embodiment of the mattress 48 is described in U.S. Provisional Application Ser. No. 63/769,453, filed Mar. 10, 2025 which is incorporated by refence in its entirety for the description of the related structures. Through the addition of the weight 49, the mattress 48 is adapted to stay in place when the patient support apparatus 10 is placed in a pelvic examination position shown in FIG. 2, the pelvic examination position providing improved access to a pelvic region when the patient support apparatus 10 is used for urological, gynecological, or obstetric examination. The patient support apparatus 10 includes a pair of handles 50, 52 which are movable between a stowed position such as that shown in FIG. 1 and a deployed position shown in FIG. 2. In the deployed position, the handles 50, 52 may be used as a labor grip for a woman laboring with the delivery of a child, or may be used as foot supports when a patient is in a supine position undergoing a pelvic examination. The mechanism and operation of the handles 50, 52 is described in detail in U.S. Patent Application Publication No. 2024/0398641A1, filed May 31, 2024 which was incorporated by reference above, the disclosure of structures suitable for the handles 50, 52 also being incorporated by refence for the description of the structures related to the handles 50, 52.

Still further, the patient support apparatus 10 includes an oxygen tank storage structure 54 which is reversible to adapt the position of an oxygen tank stored at the head end 56 of the patient support apparatus 10. An embodiment of the oxygen tank storage structure 54 is disclosed in U.S. patent application Ser. No. 19/271,229, filed Jul. 16, 2025 which is incorporated by refence in its entirety for the description of the oxygen tank storage related structures.

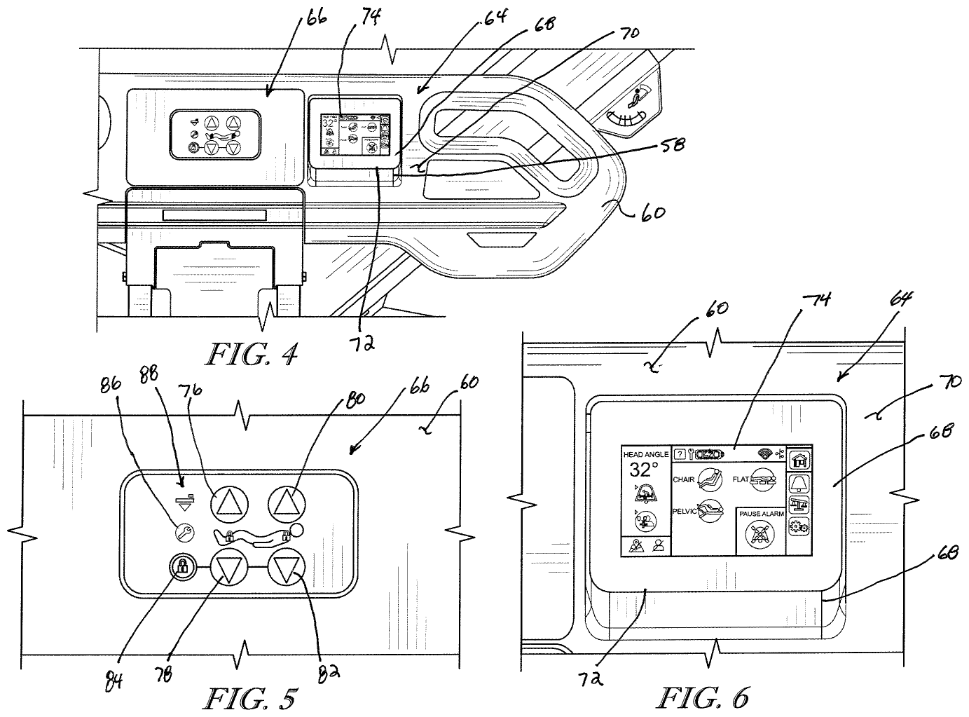

The patient support apparatus 10 further includes a pair of siderails 60, 62 as shown in FIG. 1. The siderails 60, 62 include several user interfaces, including a graphical user interface 64 and a switch panel 66 as shown in FIGS. 1-3 and 4-6. Referring to FIG. 4, the switch panel 66 is positioned in a central location on the siderail 60 and the graphical user interface 64 is positioned adjacent to the switch panel 66. The graphical user interface 64 includes a pivotable housing 68 which is pivotable relative to a body 70 of the siderail 60 so that a lower end 72 of the pivotable housing 68 may be pulled out from a cavity 58 formed in the body 70 so that a touchscreen 74 of the graphical user interface 64 is easier for a user, such as a caregiver, to see and manipulate the touchscreen 74.

The switch panel 66 includes a raise key 76 and a lower key 78 for causing powered movement of the head deck section 32 relative to the upper frame 30. The switch panel 66 also includes a thigh raise key 80 and a thigh lower key 82 for causing powered movement of the thigh deck section 36 relative to the upper frame 30. The switch panel 66 also includes a lockout key 84 which is engaged by a user to allow the keys 76, 78, 80, and 82 to be locked out. This reduces accidental activation of the raising and/or lowering of the head deck section 32 and thigh deck section 38. When the lockout key 84 is depressed while simultaneously pressing one of the keys 76, 78, 80, and 82, the respective function associated with the respective key 76, 78, 80, and 82 toggles between the state it is currently in and the opposite state. In other words, to lockout the raise key 76, a user activates the lockout key 84 and raise key 76 to cause the head deck section 32 raise function to be locked out. To unlock the raise key 76, the user again activates the lockout key 84 and raise key 76 to cause the head deck section 32 raise function to be unlocked. This functionality operates for all of the keys 76, 78, 80, and 82.

The switch panel 66 also includes a service indicator 86 that illuminates if the patient support apparatus 10 is in need of service. Additionally, a bed down indicator 88 illuminates to provide an indication that the patient support apparatus 10 is in its lowest position. Referring now to FIG. 1 and FIG. 15, a switch panel 180 is positioned on an interior surface of the siderail 62 and positioned for access by a patient. The switch panel 180 provides access to several keys 182, 184, 186, and 188, actuable by a user to cause the powered drives of the patient support apparatus 10 to move. A head raise key 182 and a head lower key 184 for causing powered movement of the head deck section 32 relative to the upper frame 30. The switch panel 180 also includes a thigh raise key 186 and a thigh lower key 188 for causing powered movement of the thigh deck section 36 relative to the upper frame 30. The switch panel 180 is duplicated on the interior surface of siderail 60 (not shown) so that the functions are accessible on either side of the patient support apparatus 10 on each siderail 60, 62 so that the functionality is available is one of the siderails 60, 62 is lowered.

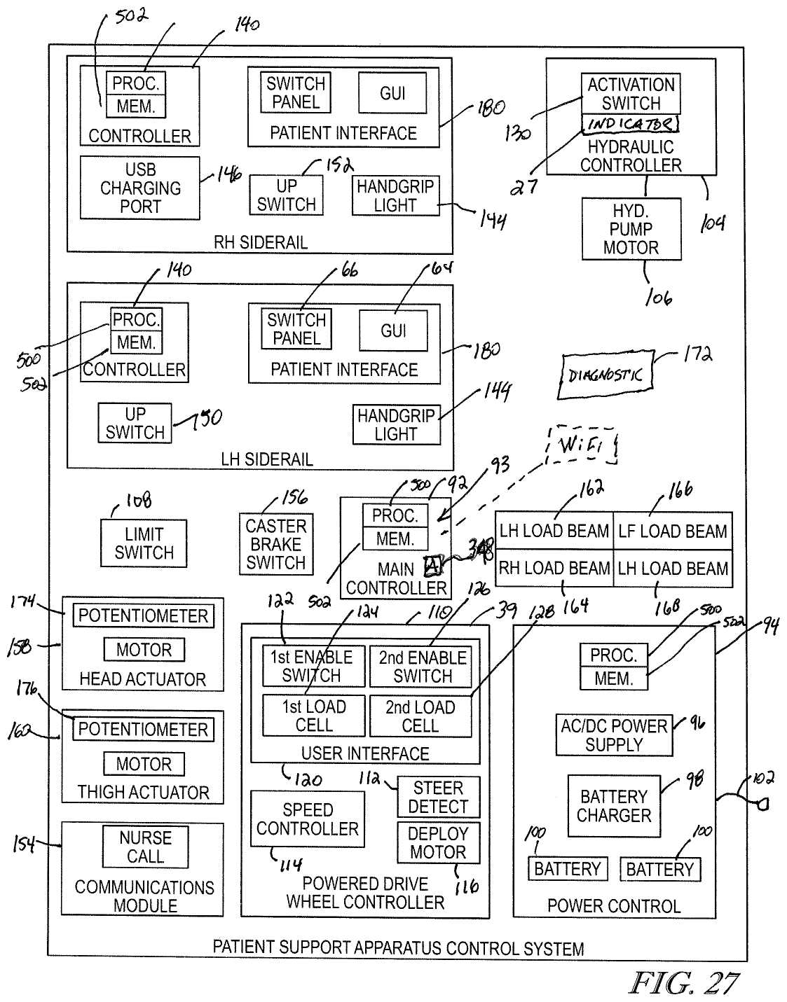

It should be noted that the patient support apparatus 10 is fully modular so that electrical options may be omitted to simplify operation and reduce costs. For example, the patient support apparatus 10 may be a fully manual device with no scale components and all of the movement being achieved by manual inputs of a user. Referring now to FIG. 27, a block diagram of a control system 90 of the patient support apparatus 10 is illustrated. The control system 90 includes a main controller 92 that comprises a system on a module 93 that provides the central logic for the patient support apparatus 10 and communicates with various optional modules of the patient support apparatus 10 as will be discussed in further detail below. When present, the main controller 92 receives power from a power control assembly 94 that includes an AC/DC power supply 96, an AC battery charger 98, and a pair of batteries 100, 100. The AC/DC power supply 96 and AC battery charger 98 each receive power directly from mains power through a power cord 102. Power consumption of the system on a module 93, which is a high performance microprocessor system which facilitates WiFi and other data connections from the patient support apparatus 10. The system on a module 93 includes an operating system, illustratively embodied as Linux® that has a feature which allows the state of the operating system executable to be written out to flash memory 502 and the processor 500 is then stopped. This reduces the current consumption of the system on a module 93 significantly. The Linux port allows the operating system state to be written out to flash memory 502 and the processor 500 stopped by executing a STOP command which is initiated after the processor has not received any inputs for a defined period of time. The system on a module 93 goes from drawing approximately 350 mA from the AC/DC power supply 96 to less than 3 mA. The processor 500 stays in this state until it is woken up by toggling an I/O pin specially programmed to perform the wakeup operation. The processor 500 then loads into the processors synchronous dynamic random-access memory the operating system image previously written out and resumes execution from where it left off. Additionally, a ‘Wake On Address’ function exists within a WiFi radio of the system on a module 93 which allows the WiFi radio transmitter and portions of the receiver to be shut down, and when the address of the WiFi port on the system on a module 93 appears on the RF channel, the WiFi radio is woken up and can answer the query. In case of a system where the system on a module 93 radio primarily originates traffic, the receiver can just be shut down to reduce current and power consumption. In this way, power from the batteries 100, 100 can be conserved until it is specifically needed.

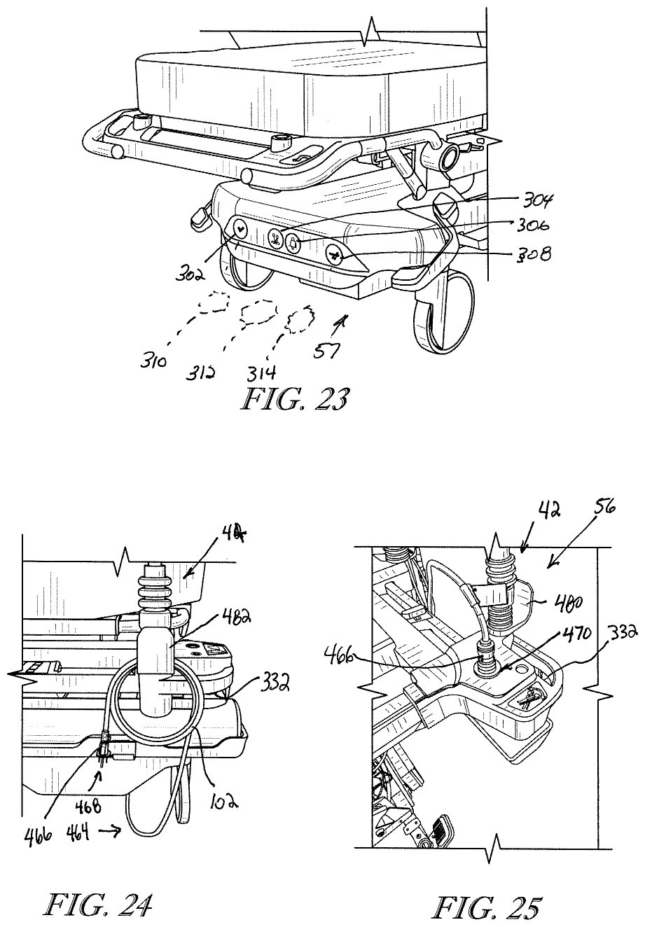

Referring to FIGS. 24 and 25, the power cord 102 may be stored when the patient support apparatus 10 is being moved in a transport mode by being draped over one of two cord holders 480, 482 positioned at the head end 56 of the patient support apparatus 10. It should be noted that during transport, the patient support apparatus 10 experiences vibrations that may cause the draped portion 464 of the power cord 102 to slacken and drag the floor. In addition, the plug 466 may be caused to slide over one of the cord holders 480, 482 and reach the floor, creating a trip hazard for a user pushing the patient support apparatus 10. As shown in FIG. 25 the prongs 468 of the plug 466 may be positioned in an opening 470 of an IV socket with an interference fit so that that the plug 466 remains secured to the patient support apparatus 10 during transport.

The power control assembly 94 also provides power directly to an optional hydraulic controller 104 that is present when the powered raise function is used with the hydraulic cylinder discussed above. The hydraulic controller 104 has an activation switch 130 that is activated when the foot pedal 26 is pushed to an activation position which thereby signals the hydraulic controller 104 to activate a hydraulic pump motor 106 which moves hydraulic fluid to the hydraulic cylinder discussed above. Once the lift arm assembly 24 achieves a fully lowered position, a low limit switch 108 is activated. While the hydraulic controller 104 operates using discrete wiring, in some embodiments, the hydraulic controller 104 may further include a processor 500 and a memory device 502, the processor 500 using instructions present on the memory device 502 to control operation of the hydraulic pump motor 106 based on the logic of the instructions to determine when to turn the hydraulic pump motor 106 on an off, based on signals from the activation switch 130 and limit switch 108. In some embodiments, the presence of the hydraulic pump motor 106 may be indicated by an optional illuminated indicator 27 shown positioned on pedal 26 in FIG. 3 and in the control system 90 in FIG. 27. The presence of the illuminated indicator 227 provides an indication to a user that the powered raise function is present.

The power control assembly 94 also provides power to a powered drive wheel controller 110 that operates the powered wheel system 39 discussed above when it is present. The powered drive wheel controller 110 includes a steer mode detection switch 112, a speed controller 114, a wheel deployment motor 116, a wheel drive motor 118, and a user interface module 120. The user interface module includes a first enable switch 122 and a first load cell 124 positioned on the push handle 42 and a second enable switch 126 and a second load cell 128 positioned on the push handle 44. To operate the powered wheel system 39, the pedal assembly 16 must be placed in the steer mode which activates the steer mode detection switch 112. This causes the wheel deployment motor 116 to move the powered wheel 40 into engagement with the floor. Once the casters 14 are placed in the steer mode, and when both the first enable switch 122 and second enable switch 126 are activated, the powered drive wheel controller 110 looks for signals from the first and second load cells 124, 128. The magnitude of the input from the load cells 124, 128 is considered by the speed controller 114 to provide a speed control signal to the wheel drive motor 118 to control the speed of the powered wheel 40. It should be noted that the powered drive wheel controller 110 operates exclusively on battery power from the batteries 100, 100.

The siderails 60, 62 each include a siderail controller 140 that communicates with the main controller 92. Each siderail controller 140 communicates with a respective patient interface 142, the graphical user interface 64, the switch panel 66, and a handgrip light 144 (shown in FIGS. 1-3, and 5). The right siderail 62 also includes a USB charging port 146. The siderails 60, 62 also each include a respective siderail up switch 150, 152 that is in independent communication with the main controller 92 to provide an indication when the respective siderail 60, 62 is in the fully raised position. The user control of the various functions of the patient support apparatus 10 will be discussed in further detail below.

The main controller 92 is also in communication with a wireless communications module 154 that is operable to communicate with a centralized communication system (not shown) to share information from the patient support apparatus 10 and to control devices that are external to the patient support apparatus 10. In some embodiments, the main controller 92 and wireless communications module is configured to communicate bed status data and alerts to nurse call systems and other systems of a healthcare facility. For example, the control system 90 may embody hardware and software similar to that disclosed in US2022/0233382 titled “TIME-BASED WIRELESS PAIRING BETWEEN A MEDICAL DEVICE AND A WALL UNIT,” published Jul. 28, 2022 and USUS2022/0313515A1 titled “WIRELESS CONFIGURATION AND AUTHORIZATION OF A WALL UNIT THAT PAIRS WITH A MEDICAL DEVICE,” published Oct. 6, 2022, each of which is incorporated by reference herein in their entirety.

In addition, the main controller 92 is in communication with a caster brake mode switch 156 that provides an indication when the casters 14 are in a fully braked position, a head actuator 158 for moving the head deck section 32, a thigh actuator 160 for moving the thigh deck section 36, four load beams 162, 164, 166, 168, a speaker 170, and a USB diagnostic port 172. The head actuator 158 and thigh actuator 160 each include a respective potentiometer 174, 176 that is also in communication with the main controller 92 to provide detailed information regarding the actual position of the head deck section 32 and thigh deck section 36 as the deck sections 32, 36 are moved relative to the upper frame 30. The load beams 162, 164, 166, 168 support the upper frame 30 to provide information about the weight and the position of a patient supported on the patient support apparatus 10, as is known in the art.

In some embodiments, when an embodiment of patient support apparatus 10 lacks the main controller 92, the four load beams 162, 164, 166, 168 may be present and a separate scale module including a simplified user interface and a dedicated scale board may be present and powered by a replaceable battery, such as a AA battery, for example.

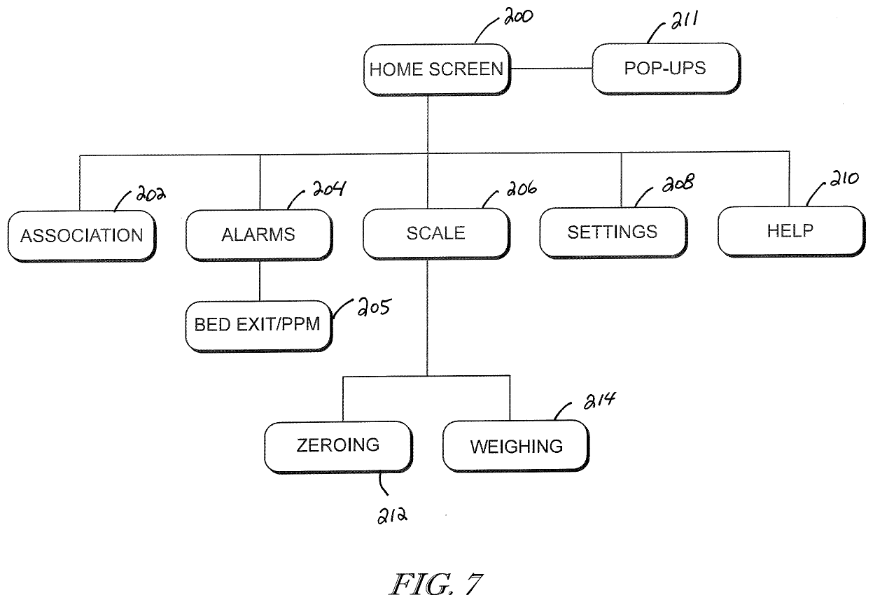

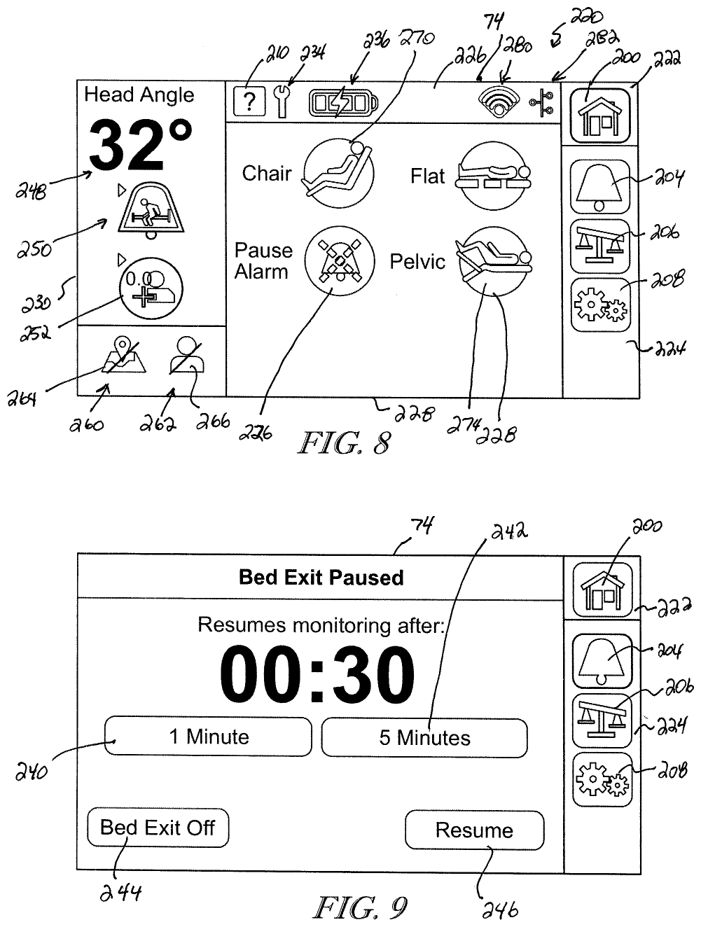

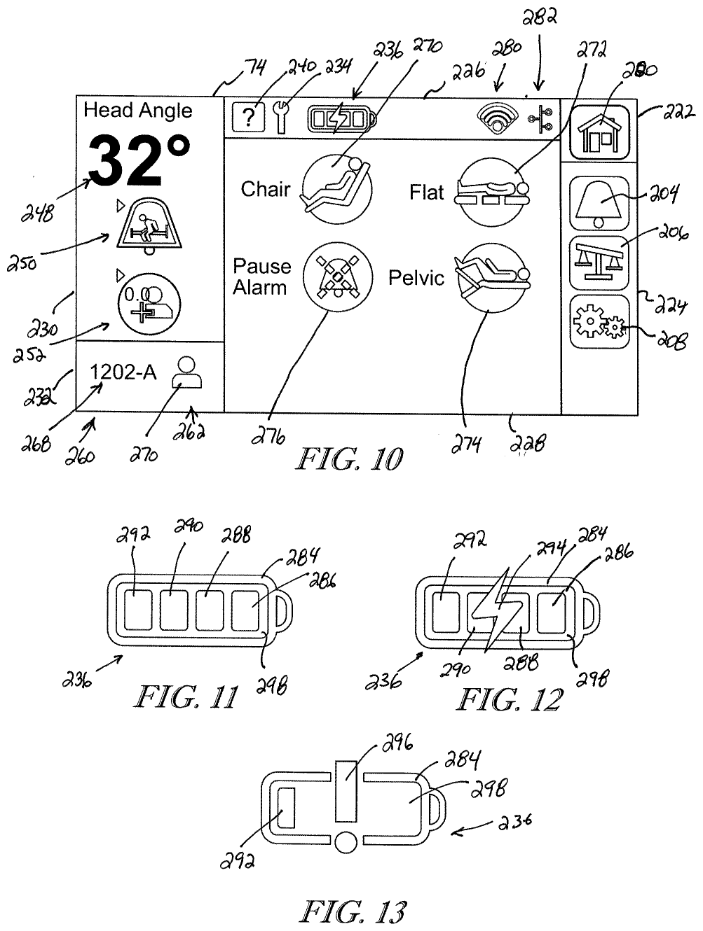

Referring now to FIG. 7, an overview of a menu structure for the graphical user interface 64 is shown to include a home screen function 200, a patient association function 202, an alarm function 204, a scale function 206, a settings function 208, a help function 210, and menu pop functionality 211. The alarm function 204 is used to set alarms for bed exit/patient position monitoring 205. The scale function 206 includes both a zeroing function 212 and a weighing function 214. Referring now to FIG. 9, an embodiment of a home screen 220 that will be displayed on the touchscreen 74 is shown to have five segments 222, 224, 226, 228, 230, 232. Segment 222 provides a home screen icon which may be illuminated in green to indicate that the home screen is being displayed and it is shown in white to indicate that the home screen is not being shown. Segment 224 is positioned at the right side of the touchscreen 74, below segment 222 and provides icons for the alarm function 204, the scale function 206, and the settings function 208. The icons 204, 206, 208 each illuminate in green when activated, but appear in white when not activated. Segment 226 is positioned at the top of the touchscreen 74 and an icon that is associated with the help function 210, another is associated with a service indication 234, and a third is associated with a battery status 236. Two additional icons 280 and 282 are associated with a first wireless connection status and a nurse call connectivity status, respectively.

Segment 228 is positioned below segment 226 and to the left of segments 222 and 224. Segment 228 is a general information segment that changes when various functions are chosen, as will be shown in additional figures below. However, when the home screen function 200 is selected, segment 228 displays icons that may be used to perform functions related to the operation of the patient support apparatus 10 in normal operation. A chair icon 270 may be touch activated by a user to move the patient support apparatus 10 to a chair position where the head deck section 32 and thigh deck section 36 are raised relative to the upper frame 30 and the foot deck section 38 is lowered relative to the thigh deck section 38. In some embodiments, the foot deck section 38 may be powered to allow it to be lowered by activating the chair icon 270. In other embodiments, the foot deck section 38 may be lowered manually. The pelvic icon 274 may be touch activated to cause the thigh deck section 36 to be raised and the foot deck section 38 to be lowered to achieve a pelvic position providing a position that eases access to the pelvic region of a patient supported on the patient support apparatus 10. The flat icon 272 may be touch activated to cause the head deck section 32, thigh deck section 36, and foot deck section 38 to be moved to a flat configuration. The pause alarm icon 276 may be activated to temporarily pause a bed exist/patient position monitoring alarm to reposition a patient. Once paused, the touchscreen 74 displays the information shown in FIG. 9 which provides a countdown timer for thirty seconds as indicated in FIG. 9. The duration may be changed by touch activating the 1 Minute button icon 240 or the 5 Minutes button icon 242. Additionally, touch activation of the Bed Exit Off button icon 244 will turn the bed exit alarm function 204 off and touch activation of the resume button icon 246 will end the Pause Alarm function immediately. If the patient exits the patient support apparatus 10 while the Pause Alarm function is active, the pop-up screen shown in FIG. 9 will be displayed while the control system 90 monitors for the patient to return to the patient support apparatus 10. Once the patient is sensed to have returned, the control system 90 will attempt to re-arm the alarm function 204 in the previously set mode. Additional pop-up screens may be displayed to assist with or confirm changes in the settings of the alarm function 204.

The segment 230 is positioned on the left side of the touchscreen 74 and displays the angle that the head deck section 32 is raised relative to the upper frame 30 at indicator 248, the alarm mode chosen at indicator 250, and the scale being zeroed at indicator 250. The indicator 248 is continuously displayed on the home screen function 200 and is directly indicated from the potentiometer 174. The scale zeroed indicator 252 is only present when the scale function 206 of the patient support apparatus 10 has been zeroed and a new patient has not been placed on the patient support apparatus 10. This provides an indication to a caregiver that a new patient may be positioned on the patient support apparatus 10. The alarm mode indicator 250 displays one of three modes that are chosen when the alarm function 204 icon is selected. The modes include a position mode, a bed exiting mode, and an out of bed mode. These modes are displayed in green when they are active and are displayed in yellow when they are suspended.

The segment 232 relates to the patient association function 202. The icon 264 in a first position 260 is a room not associated indicator, indicating that the patient support apparatus 10 has not been associated with a specific room or location within a facility. The icon 266 in a second position 262 is a patient identity not verified indicator. When the icon 264 in position 260 is flashing, a patient is assigned, but not verified, or the bed has rejected the patient identity. Referring now to FIG. 13, the text 268 in the first position 260 indicates that the room association is complete and the chair icon 270 in the second position 262, normally displayed in green, indicates that the patient has been successfully associated with the patient support apparatus 10.

Referring again now to FIGS. 1 and 23, the patient support apparatus 10 includes three distinct indicators of the status of functions of the patient support apparatus 10 that may be determined when viewing the patient support apparatus from a distance. The indication system 300 includes four different icons 302, 304, 306, 308 that are positioned at a foot end 57 of the base assembly 12, three separate icons 310, 312, 314 that are illuminated on the floor near the foot end 57 of the base assembly 12, and a light bar 316 positioned on each siderail 60, 62. The icon 302 is illuminated in two different colors to indicate the status of the charge of the batteries 100, 100 for the patient support apparatus 10. When the icon 302 is illuminated in green, it indicates that the batteries 100, 100 have at least 50% of the total charge. When the icon 302 is illuminated in amber, it indicates that the batteries 100, 100 have less than 50% of the total charge. The icon 310 is a projection that corresponds to the icon 302.

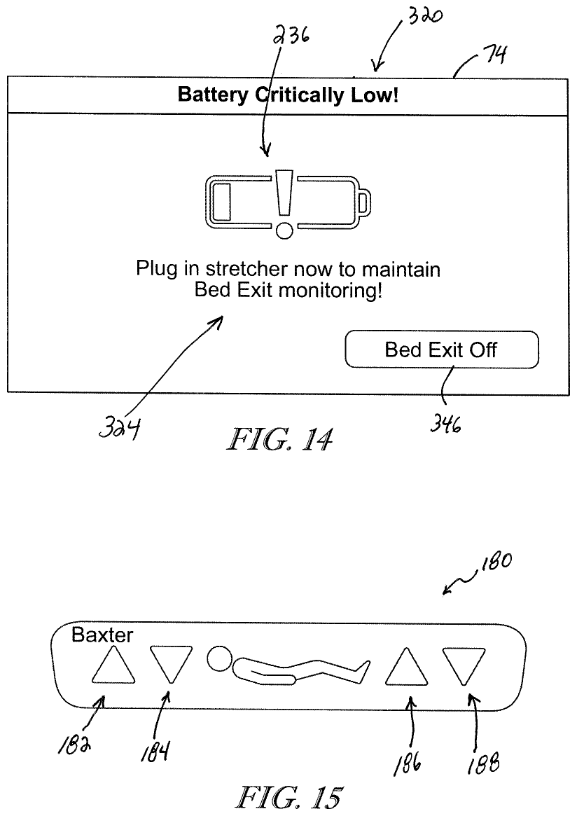

The status of the batteries 100, 100 is also provided in additional detail on the home screen 200 with reference to the battery status icon 236. In the illustrative embodiment, the battery status icon 236 has an outline 284 in the shape of an AA battery and four segments 286, 288, 290, and 292 that fill the interior of the outline 284. The four segments 286, 288, 290, and 292 provide a quick visual indication to a user of the level of charge in the batteries 100, 100. If the batteries are 100% charged, all four segments 286, 288, 290, and 292 will illuminate green. If the batteries are at least 75% charged, then segments 288, 290, and 292 will illuminate green and segment 286 will not be illuminated, but rather the space will appear the same color as a background 298. If the batteries 100, 100 are more than 50% charged but no 75% charged, then segments 290, and 292 will illuminate green and segments 286, and 288 will not be illuminated. If the batteries are greater than 25% charged but less than 50% charged the segment 292 will illuminate yellow and the other segments 286, 288, and 290 will not illuminate. If the batteries 100, 100 are less than 25% charged but still maintain a charge the battery status icon 236 will appear as shown in FIG. 15 where segment 292 will illuminate red and the other segments 286, 288, and 290 will not illuminate. In addition, a critical icon 296, illustratively in the form of an exclamation mark, will also illuminate over the outline 284. In this way, a user has an indication of the state of the batteries 100, 100 at multiple charge levels. Turning now to FIG. 14, a charging indicator 294, illustratively embodied as a lightening bolt, is illuminated over top of the battery outline 284 when the batteries 100, 100 are being charged. The charging indicator 294 is present in all of the states of charging discussed above when the batteries 100, 100 are receiving a charge, and the critical icon 296 is omitted and replaced by the charging indicator 294 when the batteries 100, 100 are less than 25% charged but still maintain a charge.

If the critical charge level indicated by FIG. 13 is reached when the patient support apparatus 10 is unplugged and the alarm function 204 is active, the touchscreen 74 will transition to the screen 320 shown in FIG. 14 where the battery status icon 236 is displayed in the form shown in FIG. 13, text bar 322 is displayed indicated that the batteries 100, 100 are critically low, and a prompt 324 is provided to have the user plug the patient support apparatus 10 in to maintain the monitoring of the alarm function 204. A touch button 346 is also provided to allow a user to turn the alarm function 204 off, rather than plug the patient support apparatus 10 into mains power. In this way, the user is given notice of the potential failure of the alarm function 204 and the option to either mitigate the lack of battery power or end the alarm function 204 monitoring.

The icon 304 illuminates blue when the bed exit alarm function 204 is off and not illuminated when the bed exit alarm function 204 is active. The icon 306 is illuminated in one of several states when the alarm function 204 is active. It is illuminated in green when a patient is in the patient support apparatus 10 and the alarm function is armed, but not alarming. The icon 306 is illuminated in amber and flashing when the alarm function 204 is active and in an alarm state. The icon 306 is illuminated in amber and solid when the alarm function 204 is paused. The icon 312 is illuminated in whatever the resulting state of the icons 304 and 306 are so that the status of the alarm function 204 is readily determined. The icons 308 and 314 correspond and are illuminated in green when the patient support apparatus 10 is in the lowest position, specifically when the lift arm assembly 24 is in the lowest possible position. The icons 308 and 314 are illuminated in amber at any other time.

The light bar 316 operates in four different states: it illuminates in a blue color when the alarm function 204 is off and there is no patient in the patient support apparatus 10; it illuminates in flashing amber color when the alarm function 204 is on and in an alarm condition; it illuminates in solid amber color when the alarm function 204 is paused and waiting on a patient to return; and it is off when either the alarm function 204 is on an not in alarming, or when the light bar 316 function has been turned off in a settings menu so that it does not respond to any functional condition of the patient support apparatus 10.

The scale function 206 of the patient support apparatus 10 operates in a manner similar to that disclosed in an PCT application publication number WO2016196403A1 titled “PATIENT SUPPORT APPARATUS” which is incorporated by reference herein for the general operation of the scale function. In addition, to the functionality disclosed in WO2016196403A1, the scale function 206 of the patient support apparatus 10 includes a capability of providing prompts to a user when the scale function 206 cannot be zeroed due to an incorrect position of components of the patient support apparatus 10, namely, the upper frame 30. Specifically, the control system 90 includes an accelerometer 348 positioned on the main controller 92 which is supported on the upper frame 30. The accelerometer 348 is operable to determine the level of tilt of the upper frame 30 and if the tilt exceeds an acceptable position for zeroing the weight detected by the load beams 162, 164, 166, 168 the touchscreen 74 displays the image 330 shown in FIG. 16, which includes text indicates that the upper frame 30 is “Not in Required Position” to perform the zeroing. The patient support apparatus 10 includes two manual releases 332, 332 positioned at a head end 56 of the patient support apparatus 10, one of which is shown on the image 330 shown in FIG. 16. The image 330 illuminates the manual release 332 in a yellow color and prompts a user to “Level Stretcher using handle” as indicated by text 334 shown in FIG. 16. The image 330 also provides a numerical indication 336 of the level of tilt of the upper frame 30 of the patient support apparatus 10 so that a user may place the upper frame 30 in an acceptable position. Image 330 is presented if the level of tilt of the upper frame 30 exceeds +/−3 degrees as such a position will not provide an acceptably accurate weight reading and the scale function 206 cannot be zeroed. A user may not proceed with zeroing the scale function 206 if the +/−3 degrees limit is exceeded but may cancel zeroing by activating a cancel button icon 338. However, if the upper frame 30 is within the required +/−3 degrees, the image 340 shown in FIG. 17 is presented. With regard to FIG. 17, the user is prompted that the upper frame 30 is “Not In Optimum Position” and the information of FIG. 16 is presented, but a user may choose to continue with zeroing the scale function 206 by activating a continue button icon 342 and the user will be permitted to perform the zeroing of the scale function 206.

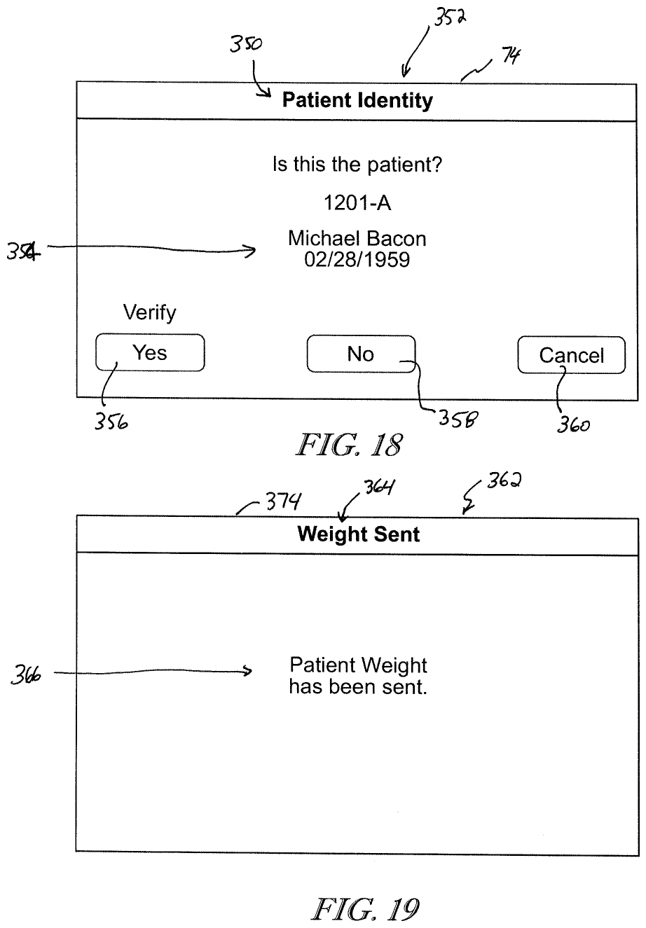

Additional capabilities of the patient support apparatus 10 include the functions shown by the images shown in FIGS. 18-22. In FIG. 18, patient association function 202 includes the function of identifying a particular “Patient Identity” as indicated by text 350 shown on an image 352 displayed on the touchscreen 74. A user is prompted with a question to verify a particular patient with text 354 which provides patient identification information. If the patient identification is correct, a user may verify that fact by activating a button icon 356 which displays the term “Yes.” If the correct patient is not shown by text 354, then a user may activate a button icon 358 which displays the term “No.” In such a case, the user will be prompted to identify correct patient information. If the user chooses to abort the patient identification step, a button icon 360 indicating “Cancel” may be activated to return to a prior screen. The functionality of FIG. 18 is related to the transfer of a patient's weight to an electronic medical record via the wireless communications module 154. If the patient is verified at image 352 of FIG. 18, then a user is notified that the patient's weight was successfully sent by an image 362 shown in FIG. 19 and displayed on touchscreen 74. The image 362 provides a text banner 364 indicating “Weight Sent” and a text prompt 366 confirming “Patient Weight has been sent.” Once the weight is sent, the touchscreen 74 returns to the home screen function 200.

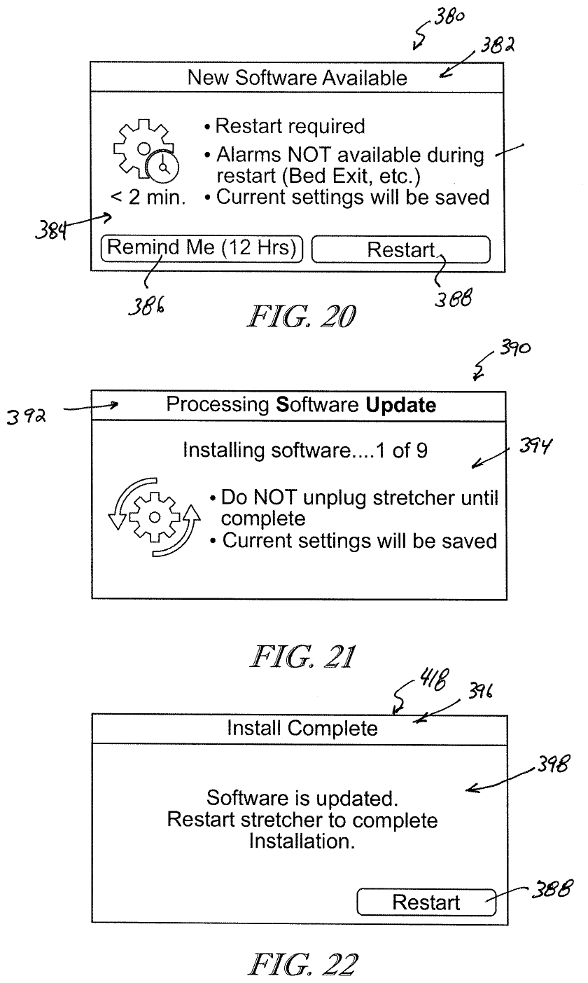

With regard to FIGS. 20-21 a progression of screens displayed on the touchscreen 74 are depicted which allow a user to be prompted to update software resident on the patient support apparatus 10. The image 380 shown in FIG. 20 provides an text indication 382 that new software is available and providing an icon 384 indicating the time for the software update to be completed along with text providing the limitations of the operation of the patient support apparatus 10 during the restart and update. A user is provided with an option to be reminded of the update after 12 hours by activating a button icon 386 or initiating the restart and software upgrade by activating a button icon 388. If button icon 386 is activated, then the touchscreen 74 returns to the home screen function 200. If button icon 388 is activated, the system advances to the status screen shown in the image 390 shown in FIG. 21 where the status of the software update is indicated by a text indication 392 and the changing status text 394. Once the installation is complete, the system advances to the status screen shown in image 418 shown in FIG. 22 where the completion of the installation is indicated by a text indication 396 and status text 398. The user may then complete the software update by activating a button icon 388. Once the patient support apparatus 10 has been restarted, the touchscreen 74 returns to the home screen function 200.

When the terms “a” or “an” or the phrases “one or more” or “at least one” are used herein, including in the claims, they are all intended to be synonymous and mean that one or more than one of the thing recited may be present. Similarly, when the phrases “a plurality” or “two or more” or “at least two” or “a pair” are used, they are all intended to be synonymous and mean that two or more than two of the things recited may be present.

According to this disclosure, phrases of the form “at least one of A and B” and “at least one of the following: A and B” and similar such phrases, mean “A alone, or B alone, or both A and B.” Phrases of the form “at least one of A or B” and “at least one of the following: A or B” and similar such phrases, also mean “A alone, or B alone, or both A and B.” Furthermore, phrases of the form “A and/or B” also mean “A alone, or B alone, or both A and B.”

Although certain illustrative embodiments have been described in detail above, variations and modifications exist within the scope and spirit of this disclosure as described and as defined in the following claims.

1. A patient support apparatus comprising a control system comprising a power supply connectable to mains power, a battery, a battery charger, and an indication system, the indication system comprising an indicator providing the status of the battery charger and an indication of the level of charge of the battery.

2. The patient support apparatus of claim 1, wherein the indication system includes a battery charge indicator positioned aa foot end of the patient support apparatus, the battery charge indicator being illuminated in multiple states to provide a status of the battery charger.

3. The patient support apparatus of claim 2, wherein the battery charge indicator provides an indication of the level of the charge of the battery and the battery charger simultaneously.

4. The patient support apparatus of claim 1, wherein the control system includes a system on a module that has a power reduction mode that is automatically engaged through an operating system when the patient support apparatus is operated on battery power without any activity for a predetermined period of time.

5. The patient support apparatus of claim 1, wherein the indication system includes a battery charge indicator positioned at a foot end of the patient support apparatus, the battery charge indicator being illuminated in multiple states to provide an indication of the level of the charge of the battery.

6. The patient support apparatus of claim 1, wherein the indication system includes a battery charge indicator positioned at a foot end of the patient support apparatus, the battery charge indicator being illuminated in multiple states to provide an indication of whether the bed is connected to mains power.

7. The patient support apparatus of claim 1, wherein the control system further comprises a graphical user interface, the graphical user interface configured to provide a battery status indicator, the indicator having multiple display states that provide an indication of the level of charge of the battery.

8. The patient support apparatus of claim 7, wherein the battery status indicator indicates whether the battery is being charged.

9. The patient support apparatus of claim 7, wherein the multiple display states include six display states.

10. The patient support apparatus of claim 9, wherein the graphical user interface is operable to generate an alert to indicate that the battery charge level is insufficient to maintain operation of a patient alarm function.

11-20. (canceled)

21. A patient support apparatus comprising
a base assembly,
a lift arm assembly coupled to the base assembly, the lift arm assembly pivotable relative to the base assembly at a head end of the base assembly,
a deck assembly supported on the lift arm assembly, the deck assembly including, a head deck a movable relative to the lift arm assembly, thigh deck section movable relative to the lift arm assembly, and a foot deck section pivotably coupled to the thigh deck section, the foot deck section having a distal end spaced apart from the thigh deck section, and
a control system including a first user input operable, when activated by a user, to cause the head deck section to be powered to be moved to a lowered position, the thigh deck section to be powered to be moved to a raised position, and the distal end of the foot deck section to be lowered relative to the thigh deck section.

22. (canceled)

23. The patient support apparatus of claim 21, further comprising a graphical user interface, the first user input being displayed on a touchscreen of the graphical user interface.

24-30. (canceled)

31. The patient support apparatus of claim 21, wherein the patient support apparatus further comprises a mattress having a weight positioned to maintain the mattress in contact with the foot deck section when the thigh deck section is raised and the distal end of the foot deck section is lowered.

32. The patient support apparatus of claim 21, wherein the lift arm assembly is driven by an electrically powered hydraulic motor, the motor being engaged when a user pushes a foot pedal down to engage an electric switch.

33. The patient support apparatus of claim 32, wherein the foot pedal includes an indicator that is illuminated to indicate that the electrically powered hydraulic motor is present.

34. The patient support apparatus of claim 21, wherein the patient support apparatus has a central wheel for steering the patient support apparatus over the floor.

35. The patient support apparatus of claim 34, wherein the patient support apparatus further comprises a push handle that includes a grip that rotates relative to a shaft of the push handle.

36. The patient support apparatus of claim 34, wherein the central wheel includes a motor for deploying the wheel into engagement with a floor and a drive motor for causing the wheel to be rotated to drive the patient support apparatus over the floor.

37. The patient support apparatus of claim 21, wherein the patient support apparatus further comprises an oxygen tank holder positioned at a head end of the patient support apparatus.

38. The patient support apparatus of claim 21, further comprising a power cord having a plug and prongs extending from the plug and a circular receptacle formed in the patient support apparatus and sized to receive the prongs in the circular receptacle with an interference fit to secure the plug to the circular receptacle.
